(19) United States
(12) Plant Patent Application Publication
(10) Pub. No.: US 2016/0120086 P1
(43) Pub. Date: Apr. 28, 2016

(54) Daphne 'MonStrk'

(50) ;

(71) Applicant: Cesar Velazquez, Salem, OR (US)

(72) Inventor: Cesar Velazquez, Salem, OR (US)

(21) Appl. No.: 14/756,871

(22) Filed: Oct. 21, 2015

Related U.S. Application Data
(60) Provisional application No. 62/122,539, filed on Oct. 22, 2014.

(51) Int. Cl. A01H 5/00

(52) U.S. Cl. PLT/226

(57) ABSTRACT

A new and distinct Daphne cultivar named ‘MonStrk’ is disclosed, characterized by variegated leaves with a distinctive large yellow margin. Foliage is distinctively elongated. The new cultivar is a Daphne, suitable for ornamental garden purposes.

### LATIN NAME OF THE GENUS AND SPECIES

Daphne odora

### VARIETY DENOMINATION

‘MONSTRK’

### BACKGROUND OF THE INVENTION

The new cultivar is a product of a chance discovery in a commercial setting by the inventor. In 2006, the inventor, Cesar Velazquez discovered an interesting single branch mutation in a commercial planting of the unpatented parent variety Daphne odora ‘Aureomarginata’. This single branch mutation was discovered at a commercial nursery in Dayton, Oreg.

During 2006 the inventor propagated the interesting mutation by semi-hardwood cuttings at the commercial nursery in Dayton, Oreg. The initial propagation showed the mutation to be stable and additional generations were propagated, also by semi-hardwood cuttings. Approximately 7 generations have been reproduced since 2006, and have shown that the unique features of this cultivar are stable and reproduced true to type.

### SUMMARY OF THE INVENTION

The cultivar ‘MonStrk’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘MonStrk’ These characteristics in combination distinguish ‘MonStrk’ as a new and distinct Daphne cultivar:

1. Variegated leaves with a wide yellow margin.

2. Elongated foliage.

### COMPARISON TO PARENT VARIETY

Plants of the new cultivar ‘MonStrk’ are similar to plants of the parent; Daphne odora ‘Aureomarginata’ in most horticultural characteristics, however, plants of the new cultivar ‘MonStrk’ have foliage with a significantly more thicker yellow margin.

### COMMERCIAL COMPARISON

‘MonStrk’ is best compared to the commercial variety Daphne odora ‘Rogbret’ U.S. Plant Pat. No. 21,820. Plants of the new cultivar ‘MonStrk’ are similar to plants of ‘Rogbret’ in most horticultural characteristics, however, plants of the new cultivar ‘MonStrk’ have a broad, more open overall plant habit. Additionally, plants of ‘MonStrk’ produce foliage with a thicker yellow margin than foliage of ‘Rogbret’.

### BRIEF DESCRIPTION OF THE PHOTOGRAPHS

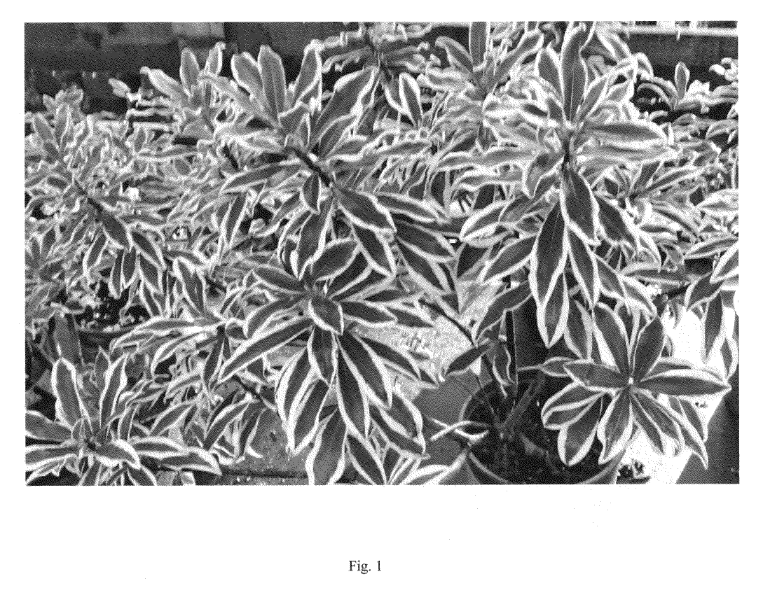

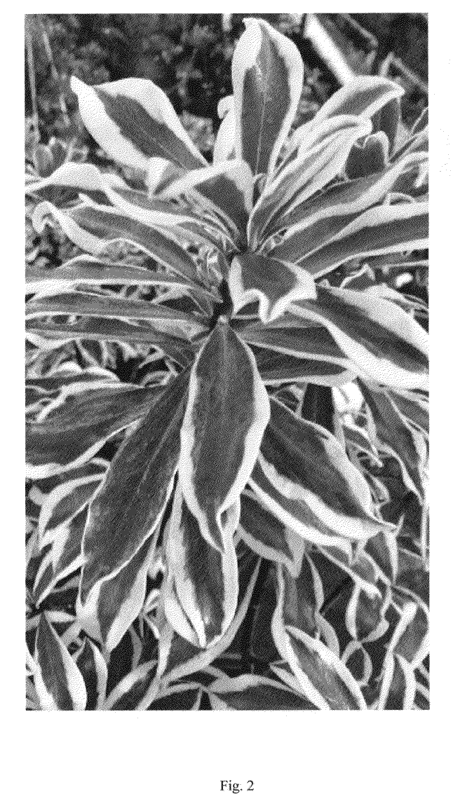

The accompanying photograph in FIG. 1 illustrates in full color a typical plant of ‘MonStrk’ grown outdoors in Oregon. FIG. 2 illustrates in full color a close up of the foliage. The plant is approximately 2 years old, and is planted in a 1 gallon pot. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘MonStrk’ plants grown outdoors in Dayton, Oreg. Plants are approximately 2 years old, in a 1 gallon nursery container. Temperatures ranged from −8° C. to 20° C. at night to 5° C. to 35° C. during the day. Measurements and numerical values represent averages of typical plant types.
Botanical classification: Daphne odora ‘MonStrk’
Propagation:
   Time to rooting.—Approximately 4 to 8 weeks at approximately 20° C. to 30° C. from semi-hardwood cuttings.
   Root description.—Fine, fibrous young roots. Older roots becoming woody.
Plant:
   Growth habit.—Upright and outwardly spreading shrub. Moderately dense.
   Height.—Approximately 50cm.
   Plant spread.—Approximately 70 cm.
   Branching characteristics.—Above average branching for a Daphne, average 5 main branches, angled approximately 45 degrees on average.
   Length of lateral branches.—Approximately 25 cm.
   Diameter of lateral branches.—Approximately 0.5 cm.
   Lateral branch strength.—Very strong, with some flexibility.
   Lateral branch color.—Young branches near RHS Yellow-Green 144A, flushed Greyed-Purple 183A. Mature branches: Near RHS Brown 200B.
   Other stem or plant characteristics.—No pubescence or lenticels observed. Texture moderately rough.
   Number of leaves per lateral branch.—Average 20 to 25.
   Age of plant described.—Approximately 2 years.
Foliage:
   Leaf.—Type: Simple Arrangement: Alternate Average Length: Approximately 9 to 12 cm. Average Width: Approximately 1.8 to 2.7 cm. Shape of blade: Elliptic Apex: Mucronate Base: Attenuate Attachment: Sessile Margin: Entire Aspect: Undulating Texture of top surface: Smooth Texture of bottom surface: Smooth Leaf internode length: Approximately 1.0 to 1.5 cm Color: Young foliage upper side: Center near RHS Green 137A. Margin near 12C and 8B. Margin width ranges from approximately 0.5 cm to 2.0 cm wide. Young foliage under side: Center near RHS Green 137A. Margin near 12C and 8B. Margin width ranges from approximately 0.5 cm to 2.0 cm wide. Mature foliage upper side: Center near RHS Green 137A. Margin near 12D and 8D. Margin width ranges from approximately 0.5 cm to 2.0 cm wide. Mature foliage under side: Center near RHS Green 137A. Margin near 12D and 8D. Margin width ranges from approximately 0.5 cm to 2.0 cm wide.
   Venation.—Type: Pinnate. Venation color upper side: Near Yellow 8D Venation color under side: Near RHS Green 138D.
   Petiole.—Not present, sessile.
Flower:
   Bloom period.—Natural Season: December to March.
   Inflorescence.—Arrangement: Single trumpet shaped flowers, composed of petaloid sepals clustered in terminal heads. Corolla not present.
Other characteristics:
   Disease and pest resistance.—Not observed to be susceptible nor resistant to normal diseases and pests of Daphne odora.
   Drought tolerance.—Some drought tolerance. Plants will not wilt or suffer significant damage if regular supplemental waterings are missed.
   Low temperature tolerance.—Hardy in to −20° C.
   High temperature tolerance.—High temperature tolerance is not exactly known. Tolerates at least 33° C.
   Fruit/seed production.—not observed to date.

What is claimed is:

1. A new and distinct cultivar of Daphne plant named ‘MonStrk’ as herein illustrated and described.
